(19) United States
(12) Patent Application Publication
(10) Pub. No.: US 2026/0232619 A1
(43) Pub. Date: Aug. 13, 2026

(54) TREATMENT OF NEUROLOGICAL DISORDERS WITH ALLICIN AND ANALOGUES THEREOF

(71) Applicant: KATHOLIEKE UNIVERSITEIT LEUVEN, Leuven (BE)

(72) Inventors: Peter DE WITTE, Kessel-Lo (BE); Lise HEYLEN, Wechelderzande (BE); Lieven LAGAE, Oud-Heverlee (BE); Daniëlle COPMANS, Holsbeek (BE); Annelii NY, Mol (BE)

(21) Appl. No.: 19/469,743

(22) PCT Filed: Mar. 28, 2024
(86) PCT No.: PCT/EP2024/058682

§ 371 Date: Sep. 26, 2025

(30) Foreign Application Priority Data
Mar. 28, 2023 (EP) 23164608.4
Jun. 6, 2023 (EP) 2317651.9

(51) Int. Cl. A61K 31/255; A61K 31/10; A61K 31/105; A61K 31/198; A61K 31/385; A61P 25/08

(52) U.S. Cl. CPC A61K 31/255; A61K 31/10; A61K 31/105; A61K 31/198; A61K 31/385; A61P 25/08

(57) ABSTRACT

The invention relates to allicin and related compounds for use in the prevention or treatment of epilepsy.

### FIELD OF THE INVENTION

The invention relates to treatment of neurological disorders such as epilepsy.

### BACKGROUND OF THE INVENTION

Epilepsy is the most common neurological disease affecting >75 million people worldwide. It is characterized by the presence of spontaneous unprovoked recurrent seizures and involves risks of comorbidities, including anxiety, depression and increased mortality. While structural brain lesions or a metabolic defect can cause epilepsy, for the majority of patients there is an underlying genetic cause Although there are over 40 approved anti-seizure medicines (ASMs) on the market to treat epilepsy, as many as 30% of patients are resistant to the effects of these drugs. Various mechanistic hypotheses exist, but what makes this drug resistant epilepsy (DRE) particularly difficult to treat is the fact that the underlying pathogenesis is variable and multifactorial within and across epilepsies and patients [Loscher & Klein (2021) CNS Drugs. 35, 935-963]

At present, no treatments are available to prevent or slow down the development of epilepsy. In fact, current treatment schedules only suppress seizures symptomatically once they occur. Fortunately, in the past few decades, the understanding of the molecular and cellular alterations that occur during epileptogenesis has markedly improved. Moreover, the introduction of novel epilepsy models and high-resolution research techniques has provided opportunities for the discovery and development of novel anti-epileptogenic drug targets, and, consequently, treatments. Such novel disease-modifying treatments act directly on the underlying disease mechanisms, potentially preventing or modifying disease onset and/or progression, and have the potential to address the current unmet medical needs, including epilepsy prevention/progression, comorbidities, and pharmacoresistance.

Given the heterogeneity of epilepsies, models for Dravet syndrome (DS), a prototype for genetic epilepsies and developmental epileptic encephalopathies, are of particular interest to study anti-epileptogenic potential of compounds. DS is a rare genetic epileptic encephalopathy accompanied with impaired neurological and psychomotor development, with onset during the first year of life. In about 80% of patients, it is caused by loss-of-function mutations in SCN1A, encoding the main Na+ channel of GABAergic neurons. Dravet syndrome is characterized by high mortality that is predominantly caused by sudden unexpected death in epilepsy [Lagae (2021). Curr Opin Neurol. 34, 213-218].

CN106727496 discloses a combination of a flavone, allicin, anisodamine and an erythritol ester (“red ancient alcohol” ester) for the treatment of epilepsy.

### SUMMARY OF THE INVENTION

In the present invention, compounds are identified that target epileptogenesis using a zebrafish model for Dravet syndrome.-Zebrafish are lower vertebrates that possess many advantages over classical rodent models including high genetic and physiological homology to humans, high fecundity, external fertilization, transparency through early larval stages that enables powerful imaging techniques, and ease of various genetic manipulations. Due to these attributes, they have successfully been deployed in large-scale, systematic drug discovery screens to identify small molecules that can suppress disease phenotypes [Patton et al. (2021) Nat Rev Drug Discov. 20, 611-628].

The invention is summarized in the following statements.

1. Alliin or a derivative thereof selected from the group consisting of allicin diallyl sulfide, diallyl disulfide, diallyl trisulfide, 1,4-dithiin and 1,2-dithiin and ajoene for use in the prevention, alleviation or treatment of a neurological disorder.

2. Allicin for use according to statement 1, in the prevention, alleviation or treatment of a neurological disorder.

3. Alliin or a derivative thereof according to statement 1 or 2, for use according to statement 1 or 2 in the prevention, alleviation or treatment of a seizure in an epilepsy patient.

4. Alliin or a derivative thereof according to statement 1 or 2 for use according to statement 3, wherein the epilepsy is Dravet syndrome.

5. Alliin or a derivative thereof according to statement 1 or 2, for use according to statement 3, wherein the epilepsy is a drug resistant epilepsy.

6. Alliin or a derivative thereof according to statement 1 or 2, for use according to statement 5, wherein the drug resistant epilepsy is an epilepsy resistant against two or more drugs selected from the group consisting of valproate, carbamazepine, levetiracetam, lamotrigine, topiramate, briveracetam, lacosamide, perampanel, and phenobarbital.

7. A method of preventing, alleviating or treating a neurological disorder in a patient, comprising the step of administering to said patient a therapeutic effective amount of alliin or a metabolite thereof selected from the group consisting of allicin, diallyl sulfide, diallyl disulfide, diallyl trisulfide, 1,4-dithiin and 1,2-dithiin and ajoene.

8. The method according to statement 7, comprising the step of administering to said patient a therapeutic effective amount of allicin.

9. The method according to statement 7 or 8, wherein the neurological disorder is epilepsy.

10. The method according to statement 9, wherein the epilepsy is Dravet syndrome

11. The method according to statement 7 or 8, wherein the epilepsy is a drug resistant epilepsy.

12. The method according to statement 11, wherein the drug resistant epilepsy is an epilepsy resistant against two or more drugs selected from the group consisting of valproate, carbamazepine, levetiracetam, lamotrigine, topiramate, briveracetam, lacosamide, perampanel, and phenobarbital.

13. A composition comprising alliin or a derivative thereof selected from the group consisting of allicin, diallyl sulfide, diallyl disulfide, diallyl trisulfide, 1,4-dithiin, 1,2-dithiin and ajoene for use in the prevention, alleviation or treatment of a seizure in an epilepsy patient.

14. The composition according to statement 13, for use according to statement 13, which comprises allicin.

15. The composition according to statement 13 or 14, for use according to statement 13, where the composition does not comprise one or more of a flavone, a flavonoid, an erythrol ester or anisodamine.

16. The composition according to any one of statements 13 to 15, for use according to statement 13, where the composition does not comprise anisodamine.

17. The composition according to any one of statements 13 to 16, for use according to statement 13, where the composition does not comprise any of a flavone, a flavonoid, an erythrol ester and anisodamine.

18. The composition according to any one of statements 13 to 16, for use according to statement 13, wherein the epilepsy is Dravet syndrome.

19. The composition according to any one of statements 13 to 15, for use according to statement 1 or 6, wherein the epilepsy is a drug resistant epilepsy.

20. The composition according to any one of statements 13 to 17, for use according to statement 19, wherein the drug resistant epilepsy is an epilepsy resistant against two or more drugs selected from the group consisting of valproate, carbamazepine, levetiracetam, lamotrigine, topiramate, briveracetam, lacosamide, perampanel, and phenobarbital.

21. A method of preventing, alleviating or treating seizure in an epilepsy patient comprising the step of administering a composition comprising alliin or a derivative thereof selected from the group consisting of allicin, diallyl sulfide, diallyl disulfide, diallyl trisulfide, 1,4-dithiin, 1,2-dithiin and ajoene.

### DESCRIPTION OF THE INVENTION

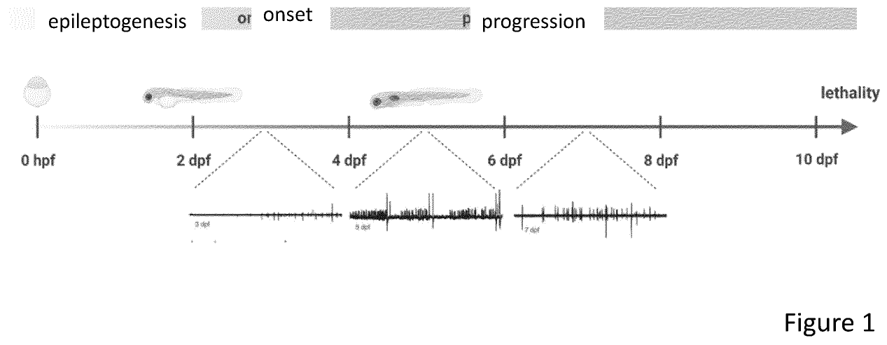

FIG. 1. Schematic overview of the timeline of epileptogenesis for the zebrafish scn1lab^{-/-} mutant model of Dravet syndrome. Starting form 3 dpf onwards, subtile epileptiform abnormalities can be observed using invasive local field potential recordings [Baraban et al. (2013) Nat Commun. 4, 2410], suggesting that epileptogenesis occurs between 0 and 3 dpf. At 5dpf, epileptiform events become more clearly pronounced. Scn1lab^{-/-} larvae die prematurely at 10-12 dpf.

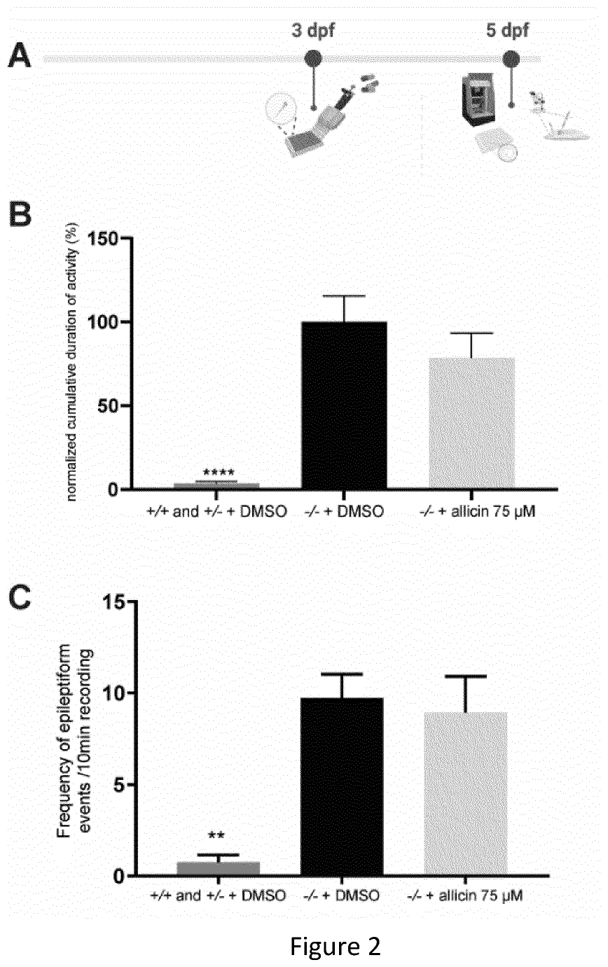

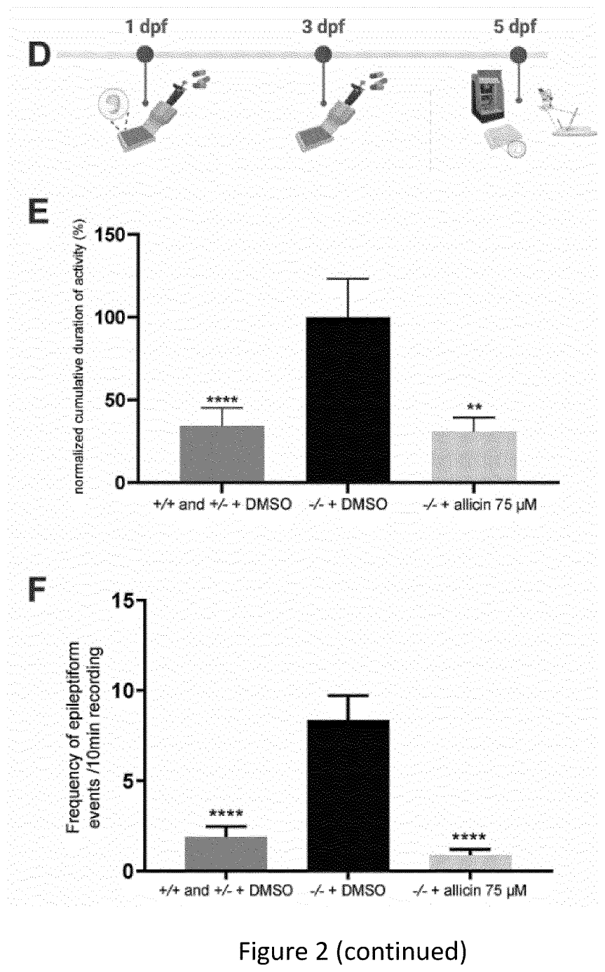

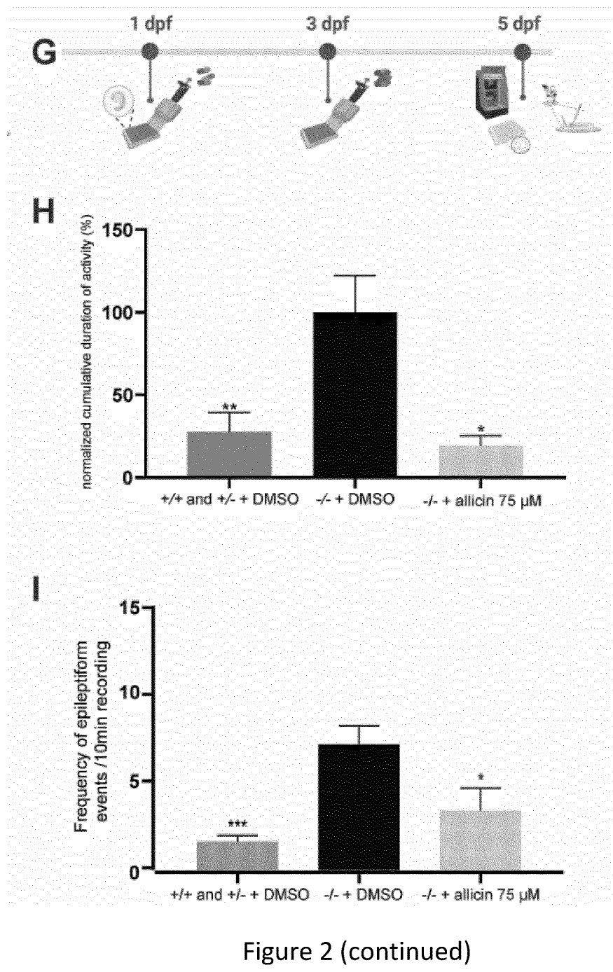

FIG. 2. Allicin treatment shows anti-epileptogenic effects. (A) Anti-seizure treatment schedule: 3 dpf scn1lab−/− larvae were exposed to the MTC of allicin or VHC control. 2 days later, abnormal locomotor behavior and epileptiform brain activity of treated larvae was analyzed using an automated tracking device (Noldus) and local field potential recordings, respectively. (B) The normalized cumulative duration of activity (%) is represented as mean±SEM. According to the treatment schedule depicted in panel A, anti-seizure effect of allicin was examined in scnLab−/− larvae at 5 dpf (n=22), and compared to VHC treated scnLab−/− larvae (n=24). VHC treated scn1Lab+/+ larvae (n=24) were included as control. (C) The average number of epileptiform events per 10-minute recording is represented as mean±SEM. Treatment schedule depicted in panel A was followed. Accordingly, electrophysiological effect of allicin was examined in scnLab−/− larvae at 5 dpf (n=15), and compared to VHC treated scnLab−/− larvae (n=15). VHC treated scn1Lab+/+ larvae (n=8) were included as control (D) Early treatment schedule: 1 dpf scn1lab−/− larvae were dechorionated and exposed to the MTC of allicin or VHC control. 2 days later, swimming medium of the larvae was replenished with identical concentrations of allicin or VHC, respectively. At 5 dpf, abnormal locomotor behavior and epileptiform brain activity of treated larvae was analyzed using an automated tracking device (Noldus) and local field potential recordings, respectively. (continued on page 6)(continued from page 5) (E) The normalized cumulative duration of activity (%) is represented as mean±SEM. According to the treatment schedule depicted in panel D, long-term effect of allicin was examined in scnLab−/− larvae at 5 dpf (n=14), and compared to VHC treated scnLab−/− larvae (n=23). VHC treated scn1Lab+/+ larvae (n=24) were included as control. (F) The average number of epileptiform events per 10-minute recording is represented as mean±SEM. Treatment schedule depicted in panel D was followed. Accordingly, electrophysiological effect of allicin was examined in scnLab−/− larvae at 5 dpf (n=18), and compared to VHC treated scnLab−/− larvae (n=14). VHC treated scn1Lab+/+ larvae (n=10) were included as control. (G) Anti-epileptogenic treatment schedule: 1 dpf scn1lab−/− larvae were dechorionated and exposed to the MTC of allicin or VHC control. 2 days later, swimming medium of all larvae was replenished with 0.1% DMSO. At 5 dpf, abnormal locomotor behavior and epileptiform brain activity of treated larvae was analyzed using an automated tracking device (Noldus) and local field potential recordings, respectively. (H) The normalized cumulative duration of activity (%) is represented as mean±SEM. According to the treatment schedule depicted in panel G, anti-epileptogenic effect of allicin was examined in scnLab−/− larvae at 5 dpf (n=8), and compared to VHC treated scnLab−/− larvae (n=17). VHC treated scn1Lab+/+ larvae (n=23) were included as control. (I) The average number of epileptiform events per 10-minute recording is represented as mean±SEM. Treatment schedule depicted in panel G was followed. Accordingly, electrophysiological effect of allicin was examined in scnLab−/− larvae at 5 dpf (n =12), and compared to VHC treated scnLab−/− larvae (n=25). VHC treated scn1Lab+/+ larvae (n=20) were included as control. For all experiments, statistical analysis was performed using one-way ANOVA. ****p≤0.0001, **p≤0.01, *p 0.05 compared to VHC treated scnLab−/− larvae.

Compounds for use in the present invention include the compound alliin from garlic and derivatives thereof such as allicin, diallyl sulfide (DAS), diallyl disulfide (DADS) and diallyl trisulfide (DAT), dithiins and ajoene.

In a specific embodiment the compound for use in the present invention is allicin.

Alliin (UIPAC name (2R)-2-Amino-3-[(S)-(prop-2-ene-1-sulfinyl)]propanoic acid) has the following structure

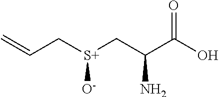

Allicin (IUPAC name S-(Prop-2-en-1-yl) prop-2-ene-1-sulfinothioate) has the following structure

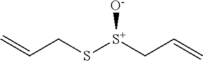

Allicin is an antioxidant activity and can react with thiol-containing proteins. Allicin has been studied for its potential to treat various kinds of multiple drug resistance bacterial infections, as well as viral and fungal infections.

Diallyl sulfide (DAS) [IUPAC name 3-prop-2-enylsulfanylprop-1-ene]has the formula CH₂=CH—CH₂—S—CH₂—CH═CH₂.

Diallyl disulfide (DADS) [IUPAC name 4,5-dithia-1,7-octadiene]has the formula CH₂=CH—CH₂—S—S—CH₂—CH═CH₂.

Diallyl trisulfide (DAT), [IUPAC name Di(prop-2-en-1-yl)trisulfane]has the formula CH₂=CH—CH₂—S—S—S—CH₂—CH═CH₂.

Dithiins 1,4-dithiin and 1,2-dithiin have the following chemical structure.

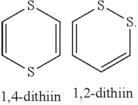

Ajoene (IUPAC name 1E)-3-(Prop-2-ene-1-sulfinyl)-1-[(prop-2-en-1-yl)disulfanyl]prop-1-ene has the following structure)

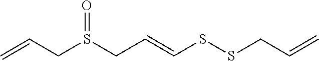

The claimed invention equally envisages the use of pharmaceutically acceptable salts, hydrates and the like of these compounds.

Optionally reducing agents and/or antioxidants can be added in the pharmaceutical formulation.

CN106727496 discloses a mixture of compounds, also comprising allicin, for the treatment of epilepsy. Other compounds in the mixture are a flavone or flavonoid, anisodamine and an erythritol ester

This Chinese patent application shows that in the mixture of these compounds, anisodamine is an essential element in the mixture, and also that the mixture without anisodamine is neither effective.

It is noted that the results of this mixture as compared to a state of the art treatment with valproate or of such an efficacy, that its accuracy is question.

In CN106727496 the use of allicin in the treatment of epilepsy is not disclosed or suggested.

Embodiments of the invention disclaim the use of anisodamine in the claimed medical use claims.

More specific embodiments further disclaim the use one or more of anisodamine, erythrol ester and a flavone or flavonoid in the claimed medical use claims.

“Neurological” disorders included but are not limited to Parkinson's disease, Alzheimer's disease, Huntington's disease, multiple sclerosis, amyotrophic lateral sclerosis, AIDS-induced dementia, epilepsy, alcoholism, alcohol withdrawal, drug-induced seizure, viral/bacterial/fever-induced seizure, trauma to the head (traumatic brain injury), spinal cord injury, hypoglycaemia, hypoxia, cerebral vascular occlusion, cerebral vascular haemorrhage, haemorrhage, an environmental excitotoxin, dementia, trauma, drug-induced brain damage, stroke/ischemia, and aging.

Seizure” refers to a brief episode of signs or symptoms due to abnormal excessive or synchronous neuronal activity in the brain. The outward effect can vary from uncontrolled jerking movement (tonic-clonic seizure) to as subtle as a momentary loss of awareness (absence seizure).

Seizure types are typically classified on observation (clinical and EEG) rather than the underlying pathophysiology or anatomy.
I Focal seizures (Older term: partial seizures)
IA Simple partial seizures—consciousness is not impaired
IA1 With motor signs
IA2 With sensory symptoms
IA3 With autonomic symptoms or signs
IA4 With psychic symptoms
IB Complex partial seizures—consciousness is impaired (Older terms: temporal lobe or psychomotor seizures)
IB1 Simple partial onset, followed by impairment of consciousness
IB2 With impairment of consciousness at onset
IC Partial seizures evolving to secondarily generalized seizures
IC1 Simple partial seizures evolving to generalized seizures
IC2 Complex partial seizures evolving to generalized seizures
IC3 Simple partial seizures evolving to complex partial seizures evolving to generalized seizures
II Generalized seizures
IIA Absence seizures (Older term: petit mal)
IIA1 Typical absence seizures
IIA2 Atypical absence seizures
IIB Myoclonic seizures
IIC Clonic seizures
IID Tonic seizures,
IIE Tonic-clonic seizures (Older term: grand mal)
IIF Atonic seizures
III Unclassified epileptic seizures

A more recent classification is published in Fisher et al. (2017) Epilepsia 58(4), 522-530.

“Epilepsy” is a condition of the brain marked by a susceptibility to recurrent seizures. There are numerous causes of epilepsy including, but not limited to birth trauma, perinatal infection, anoxia, infectious diseases, ingestion of toxins, tumours of the brain, inherited disorders or degenerative disease, head injury or trauma, metabolic disorders, cerebrovascular accident and alcohol withdrawal. A large number of subtypes of epilepsy have been characterized and categorized. The classification and categorization system, that is widely accepted in the art, is that adopted by the International League Against Epilepsy's (“ILAE”) Commission on Classification and Terminology [See e.g., Berg et al. (2010), “Revised terminology and concepts for organization of seizures,” Epilepsia, 51(4), 676-685]:

### I. Electrochemical Syndromes (Arranged by Age of Onset):

I.A. Neonatal period: Benign familial neonatal epilepsy (BFNE), Early myoclonic encephalopathy (EME); Ohtahara syndrome
I.B. Infancy: Epilepsy of infancy with migrating focal seizures; West syndrome; Myoclonic epilepsy in infancy (MEI); Benign infantile epilepsy; Benign familial infantile epilepsy; Dravet syndrome; Myoclonic encephalopathy in non-progressive disorders
I.C. Childhood: Febrile seizures plus (FS+) (can start in infancy); Panayiotopoulos syndrome; Epilepsy with myoclonic atonic (previously astatic) seizures; Benign epilepsy with centrotemporal spikes (BECTS); Autosomal-dominant nocturnal frontal lobe epilepsy (ADNFLE); Late onset childhood occipital epilepsy (Gastaut type); Epilepsy with myoclonic absences; Lennox-Gastaut syndrome; Epileptic encephalopathy with continuous spike-and-wave during sleep (CSWS), also known as Electrical Status Epilepticus during Slow Sleep (ESES); Landau-Kleffner syndrome (LKS); Childhood absence epilepsy (CAE)
I.D. Adolescence-Adult: Juvenile absence epilepsy (JAE); Juvenile myoclonic epilepsy (JME); Epilepsy with generalized tonic-clonic seizures alone; Progressive myoclonus epilepsies (PME); Autosomal dominant epilepsy with auditory features (ADEAF); Other familial temporal lobe epilepsies
I.E. Less specific age relationship: Familial focal epilepsy with variable foci (childhood to adult); Reflex epilepsies

### II. Distinctive Constellations

II.A. Mesial temporal lobe epilepsy with hippocampal sclerosis (MTLE with
II.B. Rasmussen syndrome
II.C. Gelastic seizures with hypothalamic hamartoma
II.D. Hemiconvulsion-hemiplegia-epilepsy
E. Epilepsies that do not fit into any of these diagnostic categories, distinguished on the basis of presumed cause (presence or absence of a known structural or metabolic condition) or on the basis of Primary mode of seizure onset (generalized vs. focal)

### III. Epilepsies Attributed to and Organized by Structural-Metabolic Causes

III. A. Malformations of cortical development (hemimegalencephaly, heterotopias, etc.)
III. B. Neurocutaneous syndromes (tuberous sclerosis complex, Sturge-Weber, etc.)
III. C. Tumour
III. D. Infection
III. E. Trauma

### IV. Angioma

IV.A. Perinatal insults
IV.B. Stroke
IV.C. Other causes

### V. Epilepsies of Unknown Cause

Vi. Conditions with epileptic seizures not traditionally diagnosed as forms of epilepsy per se
   VI.A. Benign neonatal seizures (BNS)
   VI.B. Febrile seizures (FS)

A more recent classification can be found in Scheffer et al. (2017) Epilepsia. 58, 512-521.

“Prevention, alleviation or/and treatment of epileptic seizures” encompasses any improvement of epilepsy related conditions such as delayed onset of seizure, less severe or shorter seizure periods, a lower frequency of seizures, a lesser sensitivity against a seizure inducing trigger.

“Dravet syndrome” is a severe form of childhood epilepsy characterized by drug-resistant seizures and numerous physical, behavioural and intellectual comorbidities. Nearly 90% of all patients with Dravet syndrome carries a mutation in the SCN1A gene (sodium channel, voltage gated, type 1 alpha subunit). encoding the main Na+ channel of GABAergic neurons. Typically, the first seizure (often triggered by fever) occurs in infancy, around the age of 3 to 6 months, in an otherwise healthy child. Later, development slows down and pharmacoresistant tonic-clonic, myoclonic, and absence-like seizures become more frequent. Patients with DS suffer from severe comorbidities, including developmental, cognitive, and behavioral deficits, as well as high mortality rates, predominantly caused by sudden unexpected death in epilepsy (SUDEP).

“Drug-resistant epilepsy (DRE)” is defined by Kwan et al. (2010) Epilepsia 52, 1069-1077, as “failure of adequate trials of two tolerated and appropriately chosen and used antiepileptic drugs (AED schedules) (whether as monotherapies or in combination) to achieve sustained seizure freedom.”

A non-exhaustive list of anti-epileptic compounds includes Paraldehyde; Stiripentol; Barbiturates (such as Phenobarbital, Methylphenobarbital, Barbexaclone; Benzodiazepines (such as Clobazam, Clonazepam, Clorazepate, Diazepam Midazolam and Lorazepam); Potassium bromide; Felbamate; Carboxamides (such as Carbamazepine Oxcarbazepine and Eslicarbazepine acetate); fatty-acids (such as valproic acid, sodium valproate, divalproex sodium, Vigabatrin, Progabide and Tiagabine); Topiramate; Hydantoins (such as Ethotoin, Phenytoin, Mephenytoin and Fosphenytoin); Oxazolidinediones (such as Paramethadione Trimethadione and Ethadione); Beclamide; Primidone; Pyrrolidines such as Brivaracetam Etiracetam Levetiracetam; Seletracetam; Succinimides (such as Ethosuximide, Phensuximide and Mesuximide); Sulfonamides (such as Acetazolamide, Sultiame Methazolamide and Zonisamide); Lamotrigine; Pheneturide; Phenacemide; Valpromide; Valnoctamide; Perampanel; Stiripentol; Pyridoxine.

Specific types of “drug resistant epilepsy” are an epilepsy resistant against two or more drugs selected from the group consisting of valproate, carbamazepine, levetiracetam, lamotrigine, topiramate, briveracetam, lacosamide, perampanel, and phenobarbital.

Previously it was shown that scn1lab−/− larvae start to develop subtile epileptiform events at 3 dpf, that become more pronounced from 5 dpf onwards (FIG. 1) [Baraban et al. (2013) Nat Commun. 4, 2410.]. This theoretically implicates that, in order to claim anti-epileptogenic effects, treatments should be administered early on (i.e., prior to 3 dpf).

Within this study, the merely symptomatic anti-seizure effects of allicin were. For this, abnormal hyperactive behavior and epileptiform brain activity was evaluated by administering allicin from 3 dpf onwards (i.e., after seizure onset, FIG. 2A). At 5 dpf, both behavioral and electrophysiological experiments did not demonstrate significant decreases in abnormal locomotor behavior or epileptiform brain activity, respectively, compared to VHC treated controls (FIG. 2B-C).

Next, early treatment of allicin was evaluated. For this, 1 dpf embryos were dechorionated and exposed to allicin for 4 days (i.e., prior to and during seizure onset, FIG. 2D). At 5 dpf, behavioral analysis demonstrated highly significant decreases in abnormal hyperactive behavior of allicin treated scn1lab−/− larvae compared to VHC controls (FIG. 2E). These results were confirmed in electrophysiological experiments, as they show that upon treatment with allicin, epileptiform events are significantly reduced to similar levels as observed in wild-type and heterozygous siblings (FIG. 2F).

Although these results indicate that allicin has to be administered early on, and more specifically prior to the onset of seizures, in order to induce significant effects, truly preventive, anti-epileptogenic effects can only be claimed when treated exclusively during the epileptogenic period (i.e., prior to seizure onset). Therefore, embryos dechorionated at 1 dpf were exposed to allicin for only 2 days (FIG. 2G), after which a washout using 0.1% DMSO occurred. Behavioral and electrophysiological experiments at 5 dpf demonstrate similar decreases in abnormal hyperactive behavior and epileptiform brain activity as were observed for the early treatment schedule, highlighting the merely anti-epileptogenic potential of allicin.

The experimental results demonstrate that allicin and analogues, such as alliin, diallyl sulfide, diallyl disulfide and diallyl trisulfide, dithiins and ajoene, are neuroprotective, disease-modifying and anti-epileptogenic agents. Since neurological diseases share pathophysiological mechanisms, the herein claimed compounds can be equally used in other neurological diseases as well.

### Examples

### Materials and Methods

### Zebrafish Model

Larval zebrafish carrying a missense loss-of-function mutation in one of the SCN1A orthologs, scn1lab, accurately mimic the epileptic phenotype observed in patients with Dravet syndrome. In fact, homozygous scn1lab−/− mutants were shown to exhibit spontaneous abnormal electrographic brain activity, hyperactivity, convulsive behavior, increased anxiety, sleep disturbances, and metabolic deficits compared to their wildtype (WT) and heterozygous sibling.

The mutant larval fish typically develop signs of epilepsy (hyperlocomotor activity, abnormal brain electrical discharges as assessed by Local Field Potential (LFP) measurements) at 4-5 dpf (days post-fertilization). This indicates that epileptogenesis occurs prior to this timeframe, specifically during the period from 0 to 3 dpf.

### Measurement of Effects of Allicin in the Zebrafish Model

When mutant larval fish were exposed during the 0-3 dpf period to non-toxic concentrations of allicin, the typical symptoms of epilepsy (as measured at 5 dpf) like hyperlocomotor activity, and abnormal brain electrical discharges (data not shown) are not present. However, this rescue effect is not present when the fish are exposed to the compound in the 3-5 dpf period. This implies that allicin has potent anti-epileptogenic activity or disease-modifying activity.

### Measurement of Effects of Allicin Related Compounds in the Zebrafish Model

The effect of a series of allicin related compounds (alliin, diallyl sulfide, diallyl disulfide, diallyl trisulfide, 1,4- and 1,2-dithiin and ajoene), disulfiram (a disulfide, used to support the treatment of chronic alcoholism) and garlic oil are tested in this zebrafish model. Mutant larval fish are exposed during the 0-3 dpf and 3-5 dpf period to non-toxic concentrations of the compounds, followed at 5 dpf by hyperlocomotor assessment of LFP measurements.

### Zebrafish Husbandry

Adult AB wild-type (WT) zebrafish (Danio rerio) and zebrafish heterozygous for the scn1Lab mutation (scn1Lab+/−), backcrossed with Tupfel longfin WT (scn1Lab+/+) were kept at 28.0° C., on a 14/10 h light/dark cycle under standard aquaculture conditions. Fertilized eggs were collected via natural spawning. The embryos and larvae were sorted and raised in petri dishes containing embryo medium (1.5 mM HEPES, pH 7.6, 17.4 mM NaCl, 0.21 mM KCl, 0.12 mM MgSO4 and 0.18 mM Ca(NO3)2) in a Peltier-cooled incubator (IPP 260, Memmert, Schwabach, Germany) at 28.0° C. on a 14/10 h light/dark cycle. Homozygous scn1Lab^{-/-} larvae at 5 dpf were selected by their dark appearance, lack of swim bladder and slight curvature in comparison to their heterozygous and WT siblings. All animal experiments have been approved by the Ethics Committee of the KU Leuven (approval number P055/2020) and by the Belgian Federal Department of Public Health, Food Safety, and Environment (approval number LA1210199).

### Compounds

Allicin, ajoene, S-allylcystein, diallyldisulfide, diallylsulfide, alliin, diethyldisulfide 3-vinyl-4H-1,2-dithiin), as well as disulfiram and standardized garlic oil are purchased from Selleck Chemicals, Sigma-Aldrich or BenChem. Stock solutions are made in DMSO and kept at −20° C. For experiments, stock solutions are diluted in Danieau's medium to achieve a final DMSO concentration of 0.1% v/v. As a vehicle (VHC) control, 0.1% DMSO in Danieau's medium is used.

Toxicity Evaluation (zebrafish)

To evaluate maximum tolerated concentration (MTC) of compounds, WT (AB strain) larvae of 4 dpf are transferred individually into a 96-well plate containing 100 μl volume per well and exposed to a certain concentration (twofold dilution series, starting from the highest soluble concentration). After 30 hours exposure, the following parameters are investigated: touch response, morphology, posture, edema, signs of necrosis, presence of swim bladder, and heartbeat. The MTC is defined as the highest soluble concentration at which no larvae died nor showed signs of toxicity or locomotor impairment at 5 dpf. When the determined MTC in AB larvae portrayed signs of toxicity in scn1lab−/− larvae, lower concentrations (twofold dilutions) are tested.

### Pharmacological Evaluation

Before performing pharmacological experiments, maximum tolerated concentration (MTC) was determined as described previously (Zhang et al., 2017): In brief, 12 larvae of 1 dpf were dechorionated and transferred individually into a 96-well plate containing 100 μl volume per well and exposed to a certain concentration (twofold dilution series, starting from the highest soluble concentration). The medium was once more replenished at 3 dpf and at 5 dpf, the following parameters were investigated: touch response, morphology, posture, edema, signs of necrosis, presence of swim bladder and heartbeat. The MTC was defined as the highest soluble concentration at which no larvae died nor showed signs of toxicity or locomotor impairment in comparison to VHC-treated control larvae. For pharmacological evaluation, larvae were treated with VHC or compounds at their respective MTC. Distinct treatment schedules were followed (FIG. 2A-D-G).

### Locomotor Activity Assessment

5 dpf zebrafish larvae were transferred individually into a 96-well plate containing 100 μl treatment medium per well. After positioning the plate inside a DanioVision box (Noldus, Netherlands), larval behavior was recorded for 10 min in the dark after a 30 min habituation period in the dark. Ethovision XT16 (Noldus) was used to quantify locomotor behavior as cumulative duration of time spent in the highly active state (s), normalized to VHC control (%). The activity state parameter quantifies typical convulsive seizure behavior (‘highly active’) according to the following settings: averaging interval of 30 samples, number equaling 2, inactive below 0.60%, and exclusion of instances below 0.20s.

### Non-Invasive Local Field Potential (LFP) Recordings

Electrographic brain activity of 5 dpf larvae was assessed by non-invasive local field potential (LFP) recordings from the optic tectum. A blunt glass electrode (soda lime glass, Hilgenberg, Germany) pulled with DMZ Universal Puller (Zeitz, Germany) to an opening of approximately 15-20 microns, connected to a high-impedance amplifier, was filled with artificial cerebrospinal fluid (124 mM NaCl, 2 mM KCl, 2 mM MgSO4, 2 mM CaCl2, 1.25 mM KH2PO4, 26 mM NaHCO₃ and 10 mM glucose) and positioned on the skin above the optic tectum of a larva embedded in 2% low-melting point agarose (ThermoScientific). The differential extracellular amplifier (DAGAN 2400, Minneapolis, MN, USA) amplified the voltage difference 10,000 times between the signal (measured by the signal electrode) and a reference electrode. The differential signal was band pass filtered at 0.3-300 Hz and digitized at 2 kHz with a PCI-6251 interface (National instruments, UK) using WinEDR software (John Dempster, University of Strathclyde, UK). A grounding electrode was used for grounding the electrical system. All three electrodes stayed connected during the recording in ACSF. Each recording lasted for 10 minutes and was performed at room temperature. Epileptiform activity was quantified and electrograms were analyzed visually using Clampfit 10.2 software (Molecular Devices Corporation, USA60) and MatLab v8.3 (The Mathworks, Inc.). Spontaneous epileptiform events were considered when the amplitude exceeded three times the background noise and lasted longer than 50 ms.

### Mouse Model

Allicin and related compounds (alliin, diallyl sulfide, diallyl disulfide, diallyl trisulfide, 1,4- and 1,2-dithiin and ajoene), disulfiram (a disulfide, used to support the treatment of chronic alcoholism) and garlic oil Compounds are equally tested in a mouse model of Dravet syndrome. This models mirrors various features of Dravet syndrome, such as spontaneous seizures, seizures provoked by hyperthermia, and premature mortality.

### Mouse Husbandry

Mice are housed together in a pathogen-free mouse facility, following standard laboratory conditions of a 14-hour light and 10-hour dark cycle. They have unlimited access to food and water, except during experiments inducing hyperthermia-induced seizures.

### Pharmacological Evaluation (Mice)

For evaluating the anti-epileptogenic or disease-modifying effect, heterozygous Dravet mice (Scn1atm1Kea 50% C57BL/6J, 50% 129S6/SvEvTac background strain) (Jackson Laboratory, Bar Harbor, ME, USA) are used that receive a daily i.p. dose of one selected compound from P3 on, using three different doses (1, 10, 50 mg/kg). Hyperthermia-induced seizures are investigated between P25-28. To that end, a rectal probe is inserted, and mice are allowed a 10-min acclimation period to both the probe and test chamber. Core body temperature is then gradually increased by 0.5° C. every 2 min, using a heat lamp controlled by a feedback temperature controller, until reaching a maximum of 42.5° C. at 60 min post-treatment. Seizure activity typically commences with myoclonic seizures, followed by generalized tonic-clonic (GTC) seizures. Video recordings of each experiment are utilized for reviewing mouse behavior.

### Statistical Analysis

GraphPad Prism 9 software (Graphpad Software Inc., San Diego, USA) was used for all statistical analysis. One-way ANOVA followed by Dunnett's multiple comparison test was used to compare means between groups. Differences between treatment and control groups were considered statistically significant if the p-value was below 0.05 (p 0.05).

1. A composition comprising alliin or a derivative thereof selected from the group consisting of allicin, diallyl sulfide, diallyl disulfide, diallyl trisulfide, 1,4-dithiin, 1,2-dithiin and ajoene for use in the prevention, alleviation or treatment of a seizure in an epilepsy patient.

2. The composition according to claim 1, for use according to claim 1, which comprises allicin.

3. The composition according to claim 1 or 2, for use according to claim 1, where the composition does not comprise one or more of a flavone, a flavonoid, an erythrol ester or anisodamine.

4. The composition according to any one of claims 1 to 3, for use according to claim 1, where the composition does not comprise anisodamine.

5. The composition according to any one of claims 1 to 4, for use according to claim 1, where the composition does not comprise any of a flavone, a flavonoid, an erythrol ester and anisodamine.

6. The composition according to any one of claims 1 to 5, for use according to claim 1, wherein the epilepsy is Dravet syndrome.

7. The composition according to any one of claims 1 to 5, for use according to claim 1 or 6, wherein the epilepsy is a drug resistant epilepsy.

8. The composition according to any one of claims 1 to 5, for use according to claim 7, wherein the drug resistant epilepsy is an epilepsy resistant against two or more drugs selected from the group consisting of valproate, carbamazepine, levetiracetam, lamotrigine, topiramate, briveracetam, lacosamide, perampanel, and phenobarbital.
